(19) United States
(12) Patent Application Publication
(10) Pub. No.: US 2009/0298102 A1
(43) Pub. Date: Dec. 3, 2009

(54) Cell Potency Assay

(76) Inventor: Ivan N. Rich, Colorado Springs, CO (US)

Correspondence Address: Donna E. Becker;Donna E. Becker, 1954 First Street, #257, Highland Park, IL, 60035, US

(21) Appl. No.: 12/463,787

(22) Filed: May 11, 2009

Related U.S. Application Data
(60) Provisional application No. 61/052,132, filed on May 9, 2008.

(51) Int. Cl. C12Q 1/66; C12Q 1/02

(52) U.S. Cl. 435/8; 435/29

(57) ABSTRACT

Cell potency assays for use with cell-based therapies, and specifically, with stem cell therapies, are provided. Cell potency assays for bone marrow cells, mobilized peripheral blood, and umbilical cord blood are provided.

### RELATED APPLICATIONS

This application claims the benefit of priority under 35 U.S.C. § 119(e) of U.S. Provisional Patent Application No. 61/052,132, filed on May 9, 2008, which is incorporated by reference in its entirety.

### TECHNICAL FIELD

Cell potency assays for use with cell-based therapies, and specifically, with stem cell therapies, are provided. Cell potency assays for bone marrow cells, mobilized peripheral blood, and umbilical cord blood are provided.

### BACKGROUND

In the field of stem cell transplantation and umbilical cord storage, there is a need for cell potency assays to determine the growth potential of the cells to be transplanted. Various international standards for the processing of cells used in transplantation have been established, but the traditional assays currently available have been unable to reliably and reproducibly achieve those standards.

To meet the current international standards, a cell potency assay would preferably exhibit the same characteristics used for drug testing including the following: accuracy, or closeness of agreement between test results and accepted reference values; sensitivity, referring to responsiveness to a stimulus or to the proportion of correctly identified samples; specificity or selectivity, referring to the proportion of negative samples correctly identified; reliability or precision, indicating an objective measure of intra- and inter-laboratory reproducibility, used as part of the validation process; relevance, or the extent to which an assay correctly predicts or measures the biological effect of interest; and robustness, or the ability of the assay to withstand changes in protocol and transferability among laboratories.

Traditionally, the colony-forming cell assay (CFC assay) had been used to examine cell populations for transplantation. The CFC assay, however, is a differentiation assay, rather than a proliferation or potency assay. The CFC assay requires manual counting of differentiated colonies, generally after 14 days in culture. The cells are allowed to differentiate and form colonies of functionally mature cells so they can then be identified morphologically according to colony type and counted manually. The traditional CFC assay does not meet the desired characteristics for a cell potency assay discussed above.

One reason the CFC assay fails to meet the standards for measuring cell potency is because the assay was not designed or developed as a cell potency assay, but rather as an investigative research tool. This has been one of the major factors contributing to the difficulty that regulatory agencies and organizations have found in providing concrete guidelines for stem cell potency assays. Since the CFC assay has been part of the experimental and applied clinical hematology community for so long, there has been a distinct complacency to consider new technologies. Yet these new technologies can provide the necessary characteristics of a cell potency assay and many other significant improvements that can benefit not only the processing laboratory and regulatory process, but ultimately the patient.

Another difficulty with the CFC assay is that it takes 14 days to perform. Successful engraftment of a stem cell product after transplantation generally takes between 14 and 21 days, but can take much longer for cord blood. Due to the time frame of the CFC assay, generally, results are available only after transplantation. This is problematic because it leaves little time, if any, for the medical team to work on an alternative therapy in the event that engraftment does not occur.

In contrast, the type of assay desired to assess potency of cell populations for transplantation is a proliferation or potency assay. While proliferation and differentiation are related, they are fundamentally different processes. Proliferation is required for the process of differentiation to occur, but differentiation is not required for the process of proliferation to occur.

According to the Code of Federal Regulations at 21 C.F.R. § 600.3(s), “[t]he word potency is interpreted to mean the specific ability or capacity of the product, as indicated by appropriate laboratory tests or by adequately controlled clinical data obtained through the administration of the product in the manner intended, to effect a given result.” According to 21 C.F.R. § 610.10, “[t]ests for potency shall consist of either in vitro or in vivo tests, or both, which have been specifically designed for each product so as to indicate its potency in a manner adequate to satisfy the interpretation of potency given by the definition in 600.3(s) of this chapter.”

A number of other international standards are also in place. According to the latest combined edition of International Standards from the Foundation for Accreditation of Cellular Therapy (FACT), the Joint Accreditation Committee of the International Society for Cellular Therapy (ISCT), and the European Group for Blood and Marrow Transplantation (EBMT), called JACIE, “laboratory processes shall include the establishment of appropriate and validated assays and test procedures of the evaluation of cell therapy products.” (Section D6.13). These standards also state that “for products undergoing manipulation that alters the final cell population, a relevant and validated assay, where available, should be employed for evaluation of the target cell population, before and after the processing procedures.” (Section D6.13.1.3). It is interesting to note that Section D6.13.1.3 has been interpreted to mean that, since the CFC assay has not been validated, it is not required to perform the CFC assay for either bone marrow or mobilized peripheral blood.

In contrast, the International Standards for Cord Blood (CB) Collection, Processing, Testing, Banking Selection and Release, by Netcord-FACT, states that the CFU (colony-forming unit) assay must be performed on a sample from each unit of cord blood prior to release for transplant. (Section D15.1.8). While a CFU assay is required, the guidelines also call for “the use of established and validated appropriate assays, standards, and test procedures for the evaluation of the CB unit” and “adequate provisions for monitoring the reliability, accuracy, precision and performance of CBB (Cord Blood Bank) Processing Facility test procedures and instruments.” (Section D14.1.1.1 and Section D14.1.1.2). Thus, for cord blood, the total number of colonies obtained from a CFC assay is mandated prior to release of the product for transplantation, but the CFC assay does not meet the requirement to demonstrate “reliability, accuracy, precision and “performance,” all of which are needed in order to validate an assay.

It is clear that the criteria for bone marrow and mobilized peripheral blood products are different from those for cord blood. There is no scientific or logical reason for this distinction, and the lack of clear standards has largely been the result of the lack of appropriate assays. The type of assay useful for assessing the potency of cell populations for transplantation is a proliferation or potency assay. While proliferation and differentiation are related, they are fundamentally different processes. Proliferation is required for the process of differentiation to occur, but differentiation is not required for the process of proliferation to occur.

There is clearly a need for a non-subjective, reliable, and reproducible instrument-based assay which can separately assess the process of proliferation from differentiation. Manual colony enumeration relies on the colony-forming ability of the cells to differentiate and mature so that the colonies can be identified and counted. This is the basis of a functional differentiation assay whereby an unidentifiable and/or undifferentiated cell, e.g. a stem cell (by definition), acquires the features of a specialized cell. This is the definition of the differentiation process, and it is the differentiation process that is addressed in the CFC assay.

Proliferation is defined as the expansion of cells by continuous division into initially two identical daughter cells. Proliferation therefore occurs prior to differentiation or a differentiation step. Without proliferation, differentiation would not occur. Differentiation is a default program requiring prior proliferation. In order to ascertain whether a stem cell product will have the potential to exhibit short- and long-term engraftment and reconstitution, the question is whether the product has the potential to proliferate. If the cells proliferate, they will also differentiate, providing circumstances in vivo allow this to occur. Even though proliferation and differentiation are related processes in the lympho-hematopoietic system, these two processes are separate and distinct. By separating the analysis of these processes, it is possible to develop a cell potency assay that addresses the need for standardized and validated regulatory requirements.

The cell potency assay for stem cell transplantation and cord blood storage takes advantage of the fact that cell proliferation and differentiation are two separate and distinct, although related, processes. By taking advantage of these different processes, it has been possible to design a cell potency assay that is non-subjective, that is standardized, and that can be subjected to validation and proficiency testing.

### SUMMARY

Cell potency assays for determining the proliferative capacity and potency of cell populations are provided. The cell potency assay may be used for any cellular-based therapy. The assays are particularly useful for assaying bone marrow, mobilized peripheral blood, and umbilical cord blood for determining the potency of the cells for transplantation. An assay method for determining the potency of a population of primitive lympho-hematopoietic cells is provided. Generally, the assay comprises the steps of: (a) incubating a cell population comprising primitive lympho-hematopoietic cells in a cell growth medium comprising fetal bovine serum having a concentration of between 0% and about 30%, and in an atmosphere having between about 3.5% oxygen and about 7.5% oxygen; (b) contacting the primitive lympho-hematopoietic cell population with a proliferation agent, the proliferation agent comprising one or more growth factors, one or more cytokines, or combinations thereof; (c) contacting the primitive lympho-hematopoietic cell population with a reagent capable of reacting with ATP and generating luminescence in the presence of ATP; and (d) detecting luminescence generated by the reagent that reacted with the ATP in the primitive lympho-hematopoietic cell population, the level of luminescence indicating the amount of ATP in the primitive lympho-hematopoietic cell population, wherein the amount of ATP indicates the proliferative capacity and, therefore, the potency of the primitive lympho-hematopoietic cells. Preferably, the cell population or sample and a reference standard of the same cells are incubated in a cell dose-dependent manner. When the intracellular ATP levels are detected, the dose response of the sample is compared to that of the reference standard in order to determine cell potency.

### BRIEF DESCRIPTION OF THE FIGURES

FIG. 1 is an ATP standard curve.

FIG. 2 is a 3-dimensional graph showing the relationship between the number of cells plated, the number of colonies generated from CFC-GEMM using a traditional colony forming assay, and the ATP production from CFC-GEMM using the HALO®-96 MeC assay.

FIG. 3 is a 3-dimensional graph showing the relationship between the number of cells plated, the ATP production from CFC-GEMM using the HALO®-96 MeC assay, and the ATP production from CFC-GEMM using the HALO®-96 SEC assay.

FIG. 4 is a 3-dimensional graph showing the relationship between the number of colonies generated from CFC-GEMM using a traditional colony forming assay, the ATP production from CFC-GEMM using the HALO®-96 MeC assay, and the ATP production from CFC-GEMM using the HALO®-96 SEC assay.

FIG. 5 is a schematic representation of the HALO®-96 stem and progenitor cell potency assay protocol.

FIG. 6 shows the cell dose response for 7 cell populations from human bone marrow showing the levels of potency for various cell populations.

FIG. 7 shows a comparison of proliferation potential between bone marrow and umbilical cord blood using HALO®-96 MeC for 7 cell populations.

FIG. 8 is a comparison of erythropoietin preparations against a reference standard to measure drug potency.

FIG. 9 is a comparison of the potency of umbilical cord blood samples.

FIG. 10 is a flow diagram illustrating the ATP-bioluminescence cell potency assay.

FIG. 11A shows the correlation between the total nucleated cell count, TNC/kg and ATP/kg in the umbilical cord blood study in Example 6. FIGS. 11B and 11C show that there was no significant correlation between absolute neutrophil count (ANC) and CFC-GEMM or HPP-SP proliferation.

FIGS. 12A and 12B show three-point cell dose response linear regressions for CFC-GEMM (FIG. 12A) and HPP-SP (FIG. 12B) that produced statistically parallel dose response lines to the reference standard in Example 6.

FIGS. 13A and 13B show three-point cell dose response linear regressions for CFC-GEMM (FIG. 13A) and HPP-SP (FIG. 13B) that did not produced statistically parallel dose response lines to the reference standard in Example 6.

FIGS. 14A and 14B show the correlation between the iATP concentration and the slope of the linear regression curves for cord blood samples that did not exhibit parallelism with the reference standard in Example 6.

FIG. 15 shows the iATP concentrations for CFC-GEMM and HPP-SP at 5,000 cells/well for all samples stored in LN₂ in Example 6.

FIG. 16 shows the cell dose response linear regression slope for each of the CFC-GEMM and HPP-SP samples in Example 6.

FIG. 17 shows the stem cell potency for each of the CFC-GEMM and HPP-SP samples in Example 6.

### DETAILED DESCRIPTION OF EMBODIMENTS

Cell potency assays provided are particularly useful for determining the proliferative capacity and, therefore, the potency, of stem cells and cord blood used for transplantation. The cell potency assays for stem cell transplantation and cord blood storage take advantage of the fact that cell proliferation and differentiation are two separate and distinct, although related, processes. By taking advantage of these different processes, it has been possible to design a cell potency assay that is non-subjective, that is standardized, and that can be subjected to validation and proficiency testing.

The process of proliferation can be correlated with a number of markers, one of which is intracellular ATP (iATP) concentration. As a biochemical marker, the iATP concentration can be calibrated against an external ATP standard. Additionally, when the concentration of ATP is the limiting substrate, as in the case of iATP, the bioluminescence produced in a luciferin/luciferase reaction measured in a plate luminometer, is directly proportional to the proliferation status of the cells.

Based on these principals, a number of stem and progenitor cell proliferation assays have been developed, each of which can be used as a platform for cell potency assays. Platforms for these assays include, for example, the HALO®-96 MeC (methylcellulose) assay; the HALO®-96 SEC (suspension expansion culture) assay, and the CAMEO™-96 STD (Standardized) assay. HALO® refers to Hematopoietic and/or Hematotoxicity Assay via Luminescence Output, and CAMEO™ refers to Colony Assay Miniaturization with Enumeration Output. The HALO®-96 MeC assay is described in greater detail in U.S. Pat. No. 7,354,729, to Rich, for High-Throughput Stem Cell Assay of Hematopoietic Stem and Progenitor Cell Proliferation and U.S. Pat. No. 7,354,730, to Rich, for High-Throughput Assay of Hematopoietic Stem and Progenitor Cell Proliferation. The HALO®-96 SEC assay is described in greater detail in co-pending U.S. patent application Ser. No. 11/561,133, filed on 17 Nov. 2006, for High-Throughput Assay of Hematopoietic Stem and Progenitor Cell Proliferation, published as U.S. Patent Application Publication No. 2007/0148668 A1. Each of these patents and patent applications is incorporated herein by reference in its entirety, as are all patents and patent applications referred to herein.

Each of these three assay platforms is briefly discussed below. The HALO®-96 MeC assay platform is designed for high-throughput hemotoxicity testing to test potential toxicity at any stage of drug development as well as a stem cell potency assay for transplantation and cord blood storage processing laboratories. HALO® is a proliferation assay. Although the target cells are grown under clonal conditions in methylcellulose, the 7 day incubation time for human peripheral blood, umbilical cord, and bone marrow cells does not allow for the cells to differentiate. At 7 days, the cells stimulated with a proliferation agent comprising one or more growth factors, one or more cytokines, or combinations thereof (provided in the form of a growth factor and cytokine cocktail) are proliferating exponentially. Little, if any, differentiation occurs at that time and, therefore, colonies of mature cells are not counted.

The HALO® assay provides for standardization of the potency assay. By measuring a biochemical marker of the proliferation process, namely iATP concentration, and relying on an instrument-based readout, the assay can be calibrated and standardized. Therefore, the assay is no longer subjective like the colony forming assay. This allows for assay validation within and between laboratories as well as proficiency testing under the auspices of independent institutes or agencies. Such standardization is not possible with a standard colony-forming assay.

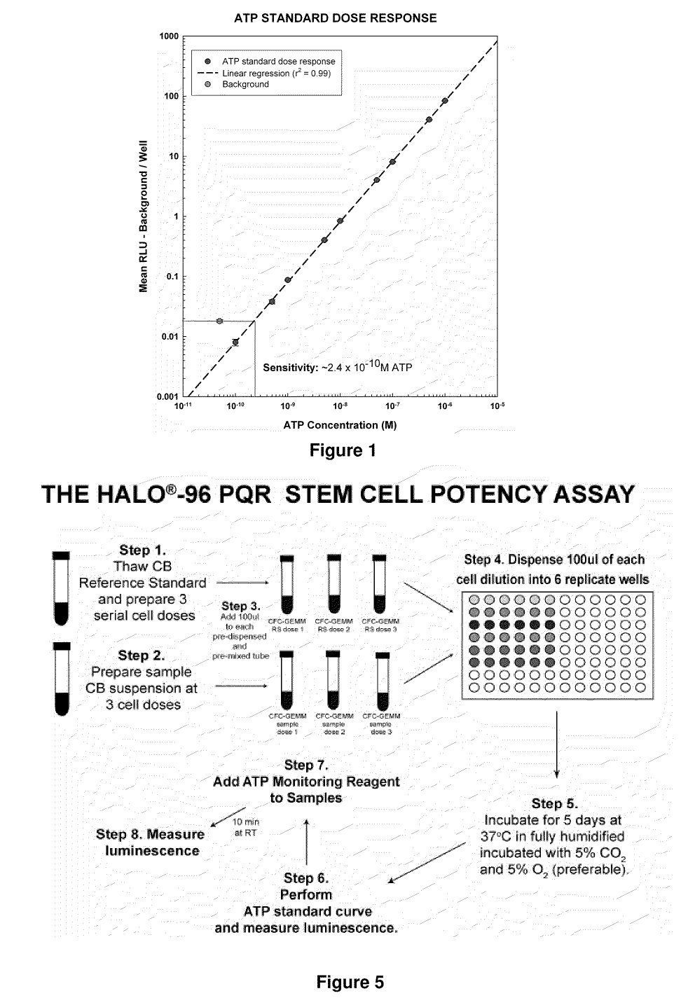

In the HALO® assay, an ATP standard curve is performed using an external ATP source prior to measuring bioluminescence as a function of iATP concentration and proliferation of the sample product. Use of an ATP standard dose response curve allows for standardization of the assay in several respects. The ATP standard curve is used to calibrate the plate luminometer and to assure that the assay reagents are working correctly. The ATP standard curve also allows standardization of the assay in that the instrument readout of non-standardized relative luminescence units (RLU) can be converted to standardized ATP concentrations (μM). An ATP standard curve is shown in FIG. 1. Luminometers from different manufactures exhibit different ranges of RLU. By using an external ATP standard, results can be compared between different plate luminometers. Finally, by performing an ATP standard curve and providing results in ATP concentrations, not only can results from different instruments be compared, but results within and between laboratories performed at different times can also be compared directly.

HALO®-96 SEC (suspension expansion culture) is a methylcellulose-free assay. The reagents are similar to those of HALO®-96 MeC, except that methylcellulose is replaced with a liquid reagent. Generally, the HALO®-96 SEC assay yields results faster than the methylcellulose assay. The cells are grown in suspension, rather than under clonal conditions, allowing cell-cell interactions to take place and reducing the lag time to the initiation of proliferation. As a result, for human cell populations, the assay generally takes 5 days to complete, rather than the 7 days for the methylcellulose format. Other species of cells may have different optimal cell culture times. For example, for canine or rat cells, the HALO®-96 SEC assay takes about 4 days while the HALO®-96 MeC assay takes about 5 days. Additionally, culturing cells under suspension expansion conditions tends to increase the sensitivity of the assay approximately two-fold over cultures grown in methylcellulose.

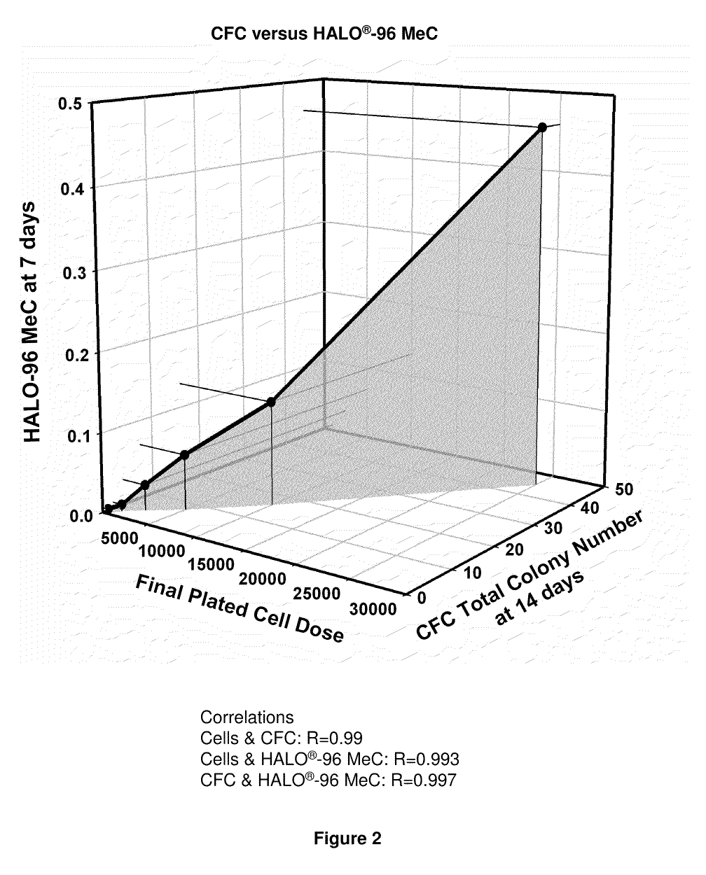

Despite these changes in culture conditions and format, there is a direct correlation between the traditional CFC assay, HALO®-96 MeC and HALO®-96 SEC as a function of cell concentration. These correlations are shown in FIGS. 2, 3, and 4. They demonstrate that the CFC assay performed at 14 days can be replaced by either HALO®-96 MeC at 7 days or HALO®-96 SEC at 5 days. Additionally, results from HALO®-96 MeC and HALO®-96 SEC also correlate with each other, and the assays can be used interchangeably.

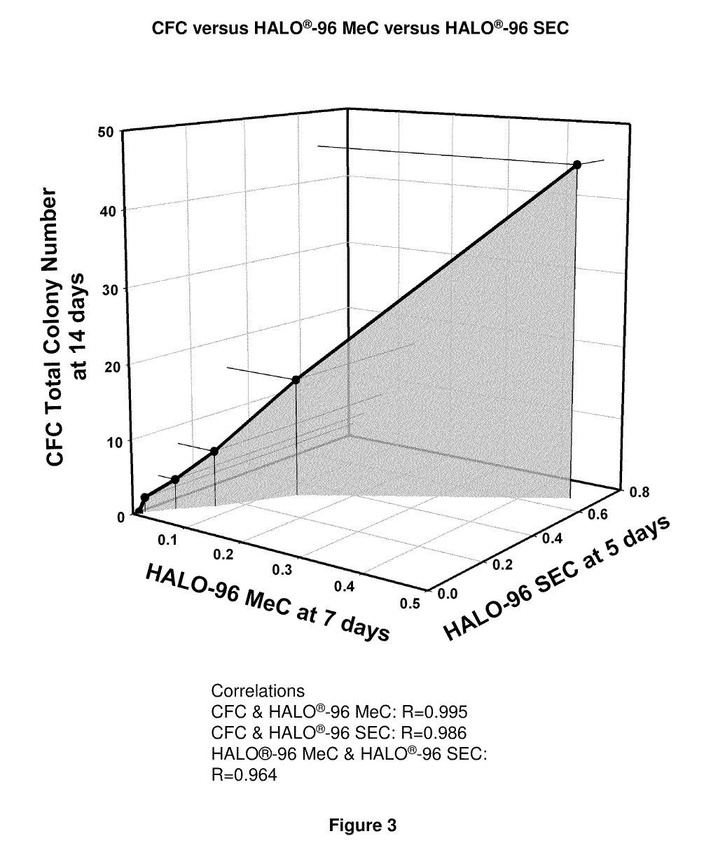

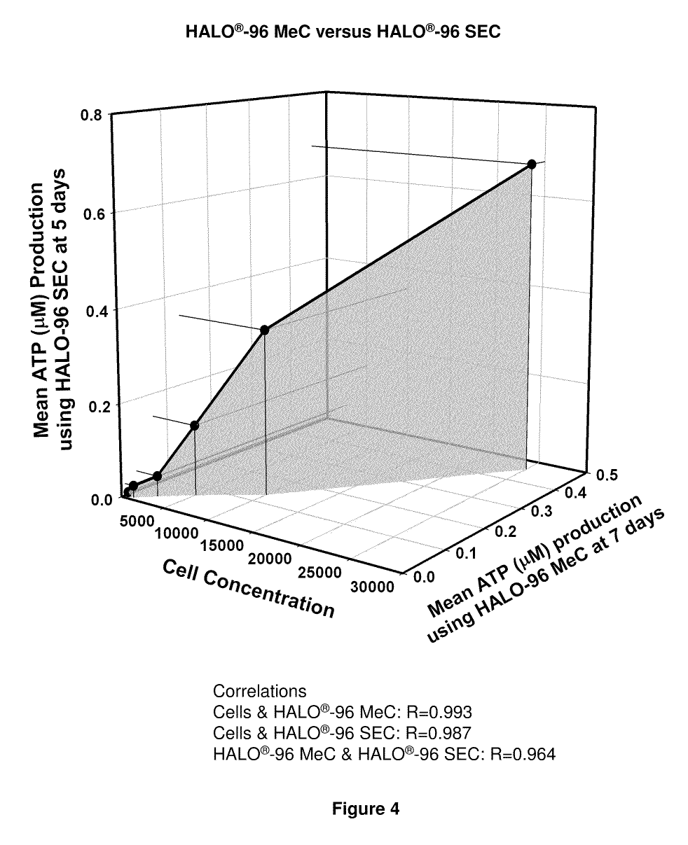

FIG. 2 is a 3-dimensional graph showing the relationship between the number of cells plated, the number of colonies generated from CFC-GEMM using a traditional colony forming assay, and the ATP production from CFC-GEMM using the HALO®-96 MeC assay. FIG. 3 is a 3-dimensional graph showing the relationship between the number of cells plated, the ATP production from CFC-GEMM using the HALO®-96 MeC assay, and the ATP production from CFC-GEMM using the HALO®-96 SEC assay. FIG. 4 is a 3-dimensional graph showing the relationship between the number of colonies generated from CFC-GEMM using a traditional colony forming assay, the ATP production from CFC-GEMM using the HALO®-96 MeC assay, and the ATP production from CFC-GEMM using the HALO®-96 SEC assay. FIGS. 2, 3, and 4, taken together, show the relationship between the traditional colony forming assay, the HALO®-96 MeC assay, and the HALO®-96 SEC assay. These data validate that there is a strong relationship between the colony forming assay and each of the HALO® platforms. These results confirm that the traditional colony forming cell assay may be replaced with a HALO® assay.

The CAMEO™-96 STD assay platform is a hybrid between the 14 day CFC assay and HALO®-96 MeC assay also performed at 14 days. CAMEO™-96 STD was designed to allow for viewing and counting of colonies to assess differentiation capability as in the CFC assay, while also allowing the measurement of proliferation as in a HALO® assay. By combining these two assays into a single system, there is the additional benefit of providing a standardized CFC assay with HALO® proliferation assay which is not possible with the traditional CFC assay alone.

CAMEO™-96 STD, like HALO®-96 MeC, can be performed in a 96-well plate using the same reagents and the same conditions as HALO®. The CFC and HALO®-96 MeC are performed in the same culture except that the incubation time extends to 14 days for CAMEO™-96 STD instead of 7 days for HALO®-96 MeC alone. Since both assays are performed under the same conditions, it is possible to measure both proliferation and differentiation in the same culture replicates.

After 14 days of incubation, the colonies produced are counted and, if required, different colony types are enumerated. The cultures are then processed to measure the iATP concentration using HALO® technology. The differentiation assay is performed first by manually counting colonies, and then a standardized proliferation assay is performed by measuring iATP-derived bioluminescence. Using CAMEO™-96 STD, both assays are performed under exactly the same conditions. As a result, the relationship between the total number of colonies counted and the iATP concentration is a direct statistical correlation producing a linear regression with a goodness of fit (r²) of close to 1. Since the measurement of iATP concentrations is performed using a calibrated and standardized procedure, the correlation allows colony counts to be expressed as iATP concentration (μM) equivalents. This correlation provides an additional benefit allowing the CFC assay to be standardized against HALO®-96 MeC. This means that there is now a CFC assay that can be standardized and validated.

Using any of these stem cell potency assay platforms, it is possible to detect lympho-hematopoietic cell populations in side-by-side cultures from the same sample. As mentioned above, additional details regarding the cell potency assay platforms are provided in U.S. Pat. Nos. 7,354,729 and 7,354,730, and U.S. Patent Application Publication No. 2007/0148668 A1, each of which is incorporated by reference in its entirety. FIG. 5 is a schematic representation of the HALO®-96 stem and progenitor cell potency assay protocol for a single stem cell population.

For a cell potency assay, there are 7 cell populations that are often used to provide useful information regarding proliferation capability of the cell population. These include two stem cell populations, three hematopoietic cell populations, and two lymphopoietic cell populations as discussed below. The High Proliferative Potential-Stem and Progenitor Cell (HPP-SP) is a primitive stem cell population that is more mature than the Long-Term Culture-Initiating Cell (LTC-IC), but more primitive than the CFC-GEMM. The HPP-SP cell population is quiescent and can either be initially “primed” to induce the cells into cell cycle or can be “fully stimulated.” The latter not only “primes” the cells, but also expands the cells into different lineages. Therefore, the HPP-SP can be used in an “Expansion Potency Assay.” When “primed” and “fully stimulated,” the HPP-SP generally exhibits the highest proliferation status of all 7 cell populations described here. The HPP-SP produces both hematopoietic and lymphopoietic cells and can be considered as occupying a stage of “stemness” that is approximately equivalent to the point at which divergence of these two lineages occurs. Like the other cell populations, the proliferation status of the HPP-SP is determined in 7 days and, therefore, does not need to rely on the 5-7 week period required for the LTC-IC. If HPP-SP cells are present, it is likely that LTC-IC are also present. The HPP-SP can be tested together with the mature multipotential stem cell population, CFC-GEMM as a duel stem cell potency assay or in combination with all 7 populations. Inclusion of the HPP-SP populations can provide valuable information on long-term engraftment and repopulation potential.

The Colony-Forming Cell-Granulocyte, Erythroid, Macrophage, Megakaryocyte (CFC-GEMM) cell is a multipotential stem cell derived from human bone marrow. This in vitro, mature, multipotential stem cell has the capability of producing cells of the granulocyte-macrophage, erythroid, and megakaryocytic lineages, but not cells of the lymphopoietic lineages. This population can be used in a single stem cell potency assay for one or a large number of samples (as in the case of cord blood centers) during pre- and post-processing screening procedures. The CFC-GEMM population is useful for short-term engraftment and reconstitution potential. It can also be combined with other hematopoietic cell populations described below. The CFC-GEMM population generally demonstrates a proliferation status lower than that of HPP-SP, but higher than the three hematopoietic progenitor cell populations.

The Burst Forming Unit-Erythroid (BFU-E) cell is a primitive erythropoietic progenitor cell population that can be included with other hematopoietic progenitor cells, and its detection combined with that of CFC-GEMM. The Granulocyte-Macrophage Colony-Forming Cell (GM-CFC) is the population that is often detected using the conventional CFC assay. Preferably, its detection may be performed in association with the CFC-GEMM, BFU-E and Mk-CFC cell populations. The Megarkayocyte Colony-Forming Cell (Mk-CFC) generally has a proliferation status similar to the BFU-E and GM-CFC, lower than the CFC-GEMM, and greater than either of the lymphopoietic lineages. Detecting the three hematopoietic lineages together can provide information about engraftment, repopulation, or reconstitution status after transplantation. T-lymphopoietic colony-forming cells (T-CFC) and B-lymphopoietic colony-forming cells (B-CFC) can be useful for monitoring lymphopoiesis after transplantation.

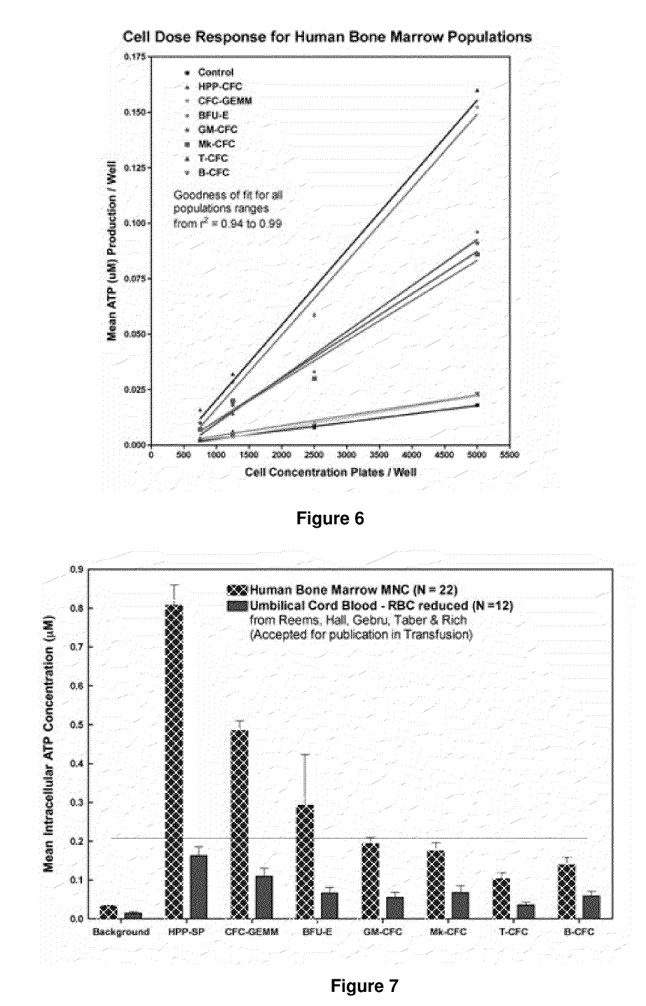

When combined together, the proliferation status of all 7 populations can provide a powerful predictive tool to monitor short- and long-term engraftment and reconstitution. The cell dose response curves for these 7 cell populations from human bone marrow are shown in FIG. 6. The slope of the cell dose response curves for each of the 7 populations is shown. The slope of the curve indicates the “primitiveness” or “stemness” of the population and, therefore, its proliferation potential. Cell concentration is plotted against mean ATP production. As seen in FIG. 6, the goodness of fit (r²) for all populations of cells is in the range of 0.94 to 0.99. Cell potency depends on the proliferation potential of the cells. Proliferation potential, in turn, is reflected in the level of ATP production. The trends of the levels of potency of various cell types discussed above are illustrated in FIG. 6.

Various embodiments of the cell potency assay are generally carried out according the following procedures. The assay is generally carried out in three basic steps that apply to each of three platforms (HALO®-96 MeC, HALO®-96 SEC and CAMEO™-96 STD). The assay platforms are described in detail in U.S. Pat. Nos. 7,354,729 and 7,354,730, and U.S. Patent Application Publication No. 2007/0148668 A1. The assay preferably utilizes pre-mixed master mixes including the assay components. These components include a serum mix, a medium, and a proliferation agent comprising one or more growth factors, one or more cytokines, or combinations thereof. In the case of a methyl cellulose based assay, methyl cellulose is also a component of the mix. The cell suspension to be assayed is adjusted to the correct concentration, and a specific volume is added to the master mix. Recommended cell concentrations are shown in Table 1. After adding the cell suspension to the master mix, the tubes are vortexed to thoroughly mix the contents, and 100 μl is dispensed into each of 6 replicate wells. This procedure usually takes about 15 to 45 minutes depending on the number of samples being assayed. The combination of assay components into a master mix is recommended in order to improve reproducibility and to reduce pipetting errors as well as the time required to set up the cell cultures.

After incubation, the cultures are processed in order to release iATP from the cells and develop the reaction to measure bioluminescence in a plate luminometer. Prior to measuring the samples, an ATP standard curve is performed. This usually takes about 15 to 20 minutes. Processing time for the samples depends on the number of samples being assayed. Generally, for a full 96-well plate, the whole procedure, including measurement, can be completed in 15 to 20 minutes.

The luminometer software can be programmed so that the RLU values produced by the instrument are automatically converted from the ATP standard curve into ATP concentrations (μM). This eliminates the need for manual calculations and plotting of the results. Most calculations including means, standard deviations, percent coefficients of variations etc., can be programmed into the software. The results can be printed out as hard copies and saved in electronic format, usually in an Excel workbook. More advanced plate luminometer software that is regulatory compliant is also available. Software packages are also available that are compliant with 21 C.F.R. (Code of Federal Regulations) Part 11 regulations.

For each platform, the procedure used follows three general steps. The first step is cell preparation. Cell samples are prepared according to a user-defined or pre-validated protocol. After ascertaining the total nucleated cell (TNC) count or the mononuclear cell (MNC) count (preferably using an electronic cell/particle counter), the cells are adjusted to a specific cell concentration. The MNC count is preferable because it provides a more accurate determination of the stem cell count. When measuring TNC, other cells such as granulocytes, which will not proliferate, are also present. The second step is the cell culture. The cell suspension is added to each tube containing pre-mixed culture reagents in a “master mix” (defined below) for each cell population to be detected. The contents of the tubes are mixed, and 100 μl is dispensed into pre-defined wells of the 96-well plate provided. The cultures are incubated for 5 days (HALO®-96 SEC), 7 days (HALO®-96 MeC), or 14 days (CAMEO™-96 STD) at 37° C. in a fully humidified atmosphere containing 5% CO₂ and, if possible, 5% O₂. The third step is measurement of proliferation by bioluminescence. To measure bioluminescence, intracellular ATP, produced as the cells proliferate, is released from the cells and acts as a substrate for a luciferin/luciferase reaction. Preferably, both lysis and luminescence reagents are provided in a single reagent. This is added using a multichannel pipette. After 10 minutes of incubation at room temperature, bioluminescence is measured in a plate luminometer. Prior to sample luminescence measurements, an ATP dose response is performed to calibrate the instrument and procedure, thereby standardizing the assay. If CAMEO™-96 STD is used, the colonies are manually enumerated prior to processing the samples for luminescence.

The cell potency assays can be used for stem cells from cord blood, as specifically described herein. The cell potency assays may also be used for other types of cells, such as bone marrow and mobilized peripheral blood, both of which are used for hematopoietic transplantation. Similarly, the same procedure is applicable to any cellular therapy, including stem cells from other tissues, embryonic stem cells. The potency assays may also be used for cells that generally do not divide or divide only under certain circumstances. For example, a potency assay can be used to measure other cellular functions or parameters, such as the production and release of various factors.

### Example 1

### Cell Potency Assay Procedure

The cell potency assays are particularly useful for determining the potency of cell populations for transplantation. Since these procedures involve the use of human cells, universal precautions for handling of human cells are recommended. If the cells being assayed are for transplantation use, the cell preparation and cell culture should be carried out using sterile conditions and in a biohazard hood.

For the traditional CFC assay, which requires 1 ml of reagents/35 mm Petri dish, normal syringes and needles are generally used. Normal syringes and needles, however, do not provide the level of accuracy desired for macro assays or for 96-well plate assays. Since small volumes are being dispensed, variations in dispensing, caused by non-calibrated or manual set pipettes, can cause large discrepancies in the results. This in turn can lead to loss of accuracy, precision, reliability and reproducibility, all of which are desired for a potency assay. Use of a positive displacement repeater pipette (preferably electronic) for dispensing operations involving methylcellulose is recommended. They are also useful for the HALO®-96 SEC assay. If electronic pipettes are available, it is recommended to use them throughout the procedure.

Step 1—Cell Preparation

Each of the bioluminescence potency assay platforms can be used with peripheral blood (normal or mobilized), bone marrow, or umbilical cord blood. In preparing the cells, erythrocytes are depleted because they can interfere with the luminescence reaction when present at high concentrations. If red blood cells are lysed, the cells are washed after lysis because the lysate contains free hemoglobin that can also interfere with the assay. For umbilical cord blood, erythrocytes are depleted using a current Hetastarch® protocol or a density gradient separation according to the manufacture's protocol. Preferably, erythrocytes constitute less than 10% of the cell suspension. Providing sufficient cells are available, subpopulations of stem and progenitor cells can be isolated and purified for use in any of the bioluminescence potency assay platforms. One method for isolating subpopulations of cells is by using magnetic cell isolation procedures (e.g. Miltenyi Biotech), as these allow for rapid isolation of different cell populations with substantial purity, viability, and yield.

Cell viability is determined and then the cell concentration can be adjusted accordingly. Cell viability can be measured using trypan blue and a hemacytometer or by flow cytometry. A viability of 85% or greater is recommended. For cryopreserved cells, a viability of 90% or greater is recommended. Cell concentration is determined using either a hemacytometer or an electronic cell/particle counter. The cell concentration is then adjusted to the desired level. Table 1 provides an example of desired cell concentrations for various cell types.

TABLE 1

| | | | Working cell | | |
|---|---|---|---|---|---|
| | | | concentration required | | |
| | Cell | | (100x Final Cell | Final cell | |
| Cell type | preparation | Cell state | Concentration) | concentration per well | |
| Bone marrow | MNC | Fresh/ | 7.5 × 10⁴-10 × 10⁵/ml | 750-10,000 | cells/well |
| | | Frozen | | | |
| Peripheral | MNC | Fresh/ | 1-10 × 10⁵/ml | 1,000-10,000 | cells/well |
| blood | | Frozen | | | |
| Umbilical cord | MNC | Fresh/ | 7.5 × 10⁴-10 × 10⁵/ml | 750-10,000 | cells/well |
| blood | | Frozen | | | |
| Bone marrow | CD34⁺ | Fresh/ | 1-5 × 10⁴/ml | 100-500 | cells/well |
| | | Frozen | | | |
| Peripheral | CD34⁺ | Fresh/ | 1-5 × 10⁴/ml | 100-500 | cells/well |
| blood, | | Frozen | | | |
| mobilized | | | | | |

| | | | | |
|---|---|---|---|---|
| Umbilical cord | CD34⁺ | Fresh/ | 2.5 × 10³/ml-5 × 10⁴/ml | 25-500/well |
| blood | | Frozen | | |

Step 2—Cell Culture

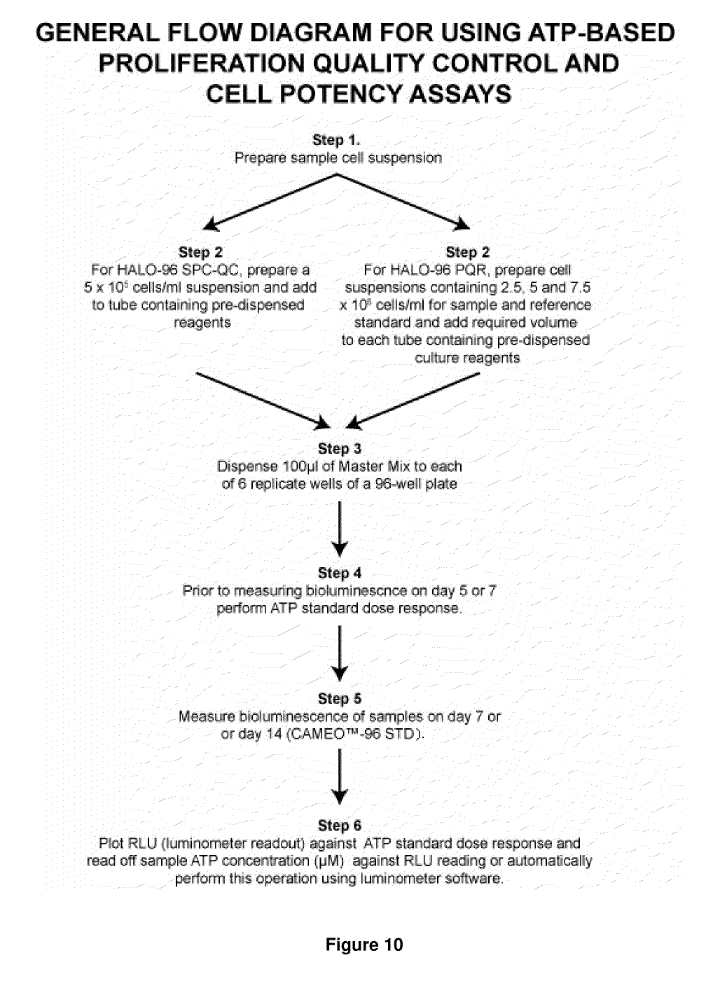

The second assay step is the cell culture. FIG. 10 shows a flow diagram for performing the ATP bioluminescence proliferation assay using any of the assay platforms described herein.

1. Tubes containing 900 μl of pre-dispensed and pre-mixed master mixes are supplied. The number of tubes depends on the number of samples and populations that can be performed using the assay kit. The contents of the tubes are supplied frozen. A number of tubes equal to the number of samples to be analyzed is thawed either in a 37° C. water bath or at room temperature.

The master mix contains the following components: 11.1 mg/ml bovine serum albumin (BSA), 0.055 mg/ml fetal bovine serum (FBS), 0.011 mg/ml recombinant insulin, and 0.222 mg/ml of iron saturated transferrin. In addition, alpha-thioglycerol is added so that the final concentration in culture is 0.1 mM. Growth factors and cytokines specific for various cell populations are also added. The master mixes and growth factors are made up in IMDM.

2. The sample cell suspension concentration is adjusted so the working cell concentration is 100 fold greater than the final cell concentration in the well. For example, if the final cell concentration is to be 5,000 cells/well (5×10³ cells/well), then the working cell concentration would be 500,000 cells/ml (5×10⁵ cells/ml). A volume of 500-1,000 μl (0.5-1 ml) of the working cell concentration is prepared.

3. A 100 μl (0.1 ml) volume of the sample working cell concentration is added to the completely thawed master mix using a calibrated (preferably electronic) pipette. The total volume in the tube is now 1,000 μl (1 ml), but the working cell concentration is reduced 10 fold.

4. The contents of the tube are mixed thoroughly by vortexing. If using methylcellulose, the contents are left for a few minutes to settle.

5. Using a positive displacement repeater pipette, 100 μl (0.1 ml) of the Master Mix containing the cell sample is dispensed into the center of each of 6 replicate wells across the plate in rows. The first sample is added to row A1 to A6, the second sample to row B1 to B6, etc. By dispensing only 100 μl (0.1 ml) of the master mix into each well, the sample working cell concentration is decreased a further 10 fold, thus producing the final cell concentration desired per well. Using a positive displacement repeater syringe to dispense these small volumes of methylcellulose-containing master mix is recommended to ensure accuracy.

6. If not all of the 96-wells have been used, the empty wells can be covered with a sterile adhesive foils.

7. The culture plate is transferred to a 37° C., fully humidified incubator containing an atmosphere of 5% CO₂. If possible, an incubator gassed with nitrogen to reduce the atmospheric oxygen concentration (21%) to 5% O₂ is used. Reducing the oxygen concentration increases the plating efficiency by reducing oxygen toxicity.

8. The incubation times used are 5 days for HALO®-96 SEC, 7 days for HALO®-96 MeC, and 14 days for CAMEO™-96 STD. Since 96-well plates have a transparent growth surface, it is possible to observe cell growth at any time using an inverted microscope. This is important for the CAMEO™-96 STD platform because mature colonies are enumerated after 14 days in culture. One drawback of a transparent growth surface is that luminescence crosstalk occurs. This is where luminescence from one well interferes with luminescence from another well. Solid white-walled plates can be used to avoid this interference and to increase the sensitivity of the assay.

9. For CAMEO™-96 STD, manual enumeration is performed prior to luminescence measurement. The release of intracellular ATP involves lysing the cells, therefore, colony counting after luminescence measurement is not possible.

Step 3—Luminescence Measurement

Prior to measuring the luminescence of the samples, an ATP standard curve is performed. Since the ATP standard curve is not performed under sterile conditions, a non-sterile, 96-well plate is used.

A. Calibration and ATP Standard Curve

1. A serial dilution of ATP standard is made from the 10 μM stock solution providing concentrations of 1 μM, 0.5 μM, 0.1 μM, 0.05 μM, and 0.01 μM using the medium provided as a diluent.

2. For each ATP concentration a total of 4×100 μl is prepared.

3. 100 μl of the supplied medium is dispensed alone into the first 4 wells of column 1 as a control to measure background luminescence.

4. Starting from the lowest ATP dilution, 100 μl is dispensed into 4 replicate wells.

5. After dispensing all of the ATP concentrations of the standard curve, 4 replicate wells, each containing 100 μl of the low and high controls provided, are also dispensed.

6. Using an 8-tip multichannel pipette, 100 μl of ATP monitoring reagent mix is added to each column and the contents are mixed. This is repeated for each column, changing tips after the contents of the wells are mixed.

7. For the ATP standard curve, no incubation time is needed.

8. The plate is placed in the luminometer, and after 2 minutes for the reaction development to occur, the plate is read.

B. Sample Measurement

1. Prior to processing the sample plate, the plate is transferred to a humidified incubator set at 22-23° C. gassed with 5% CO₂ for 30 minutes to equilibrate or to allow the plate to reach room temperature.

2. If only part of a plate is used, because there are not enough samples to fill a 96-well plate, the lid is removed under sterile conditions and a sterile adhesive foil is placed over the empty wells to avoid any contamination so that the plate can be used at a later time.

3. Using an 8- or 12-tip multichannel pipette, 100 μl of the ATP monitoring reagent mix is added to the first column. The contents are mixed and the tips are discarded.

4. This procedure is repeated for each column using new tips.

5. When the ATP monitoring reagent mix has been added to all wells, the plastic lid is replaced and the plate is incubated for 10 minutes at room temperature. During this time, cell lysis occurs and the luminescence signal becomes stabilized.

6. The plate is transferred to the luminometer and luminescence measurement is performed.

C. Automation of Results

The majority of plate luminometers are controlled by software installed on a computer. Some plate luminometers are “stand-alone” instruments and do not require a separate computer. Whether or not a computer is required, the software can generally be programmed so that sample RLU values can automatically be converted to standardized ATP concentrations from the external ATP standard dose response. Additionally, most statistical calculations can be performed, printed in tabular and graphical form, and stored.

### Example 2

### Defining Release Criteria for Umbilical Cord Blood

FIG. 7 shows a comparison of proliferation potential between bone marrow and umbilical cord blood using HALO®-96 MeC on all 7 cell populations. As expected, the proliferation potential of the cell populations from bone marrow is greater than that of the cell populations from umbilical cord blood. This is thought to be due, in large part, to the differences in handling of the two types of cell sources. Cells from bone marrow are generally harvested and used rapidly, while cord blood is generally cryopreserved for many years before use. Comparing the data for these two sources can help define release criteria for umbilical cord blood. In FIG. 7, the horizontal line above the x-axis represents an example of an arbitrary minimum threshold that could be used to determine appropriate release criteria for cord blood. In this example, the horizontal line, or arbitrary threshold, is at least 3 standard deviations above the background. For example, intracellular ATP values for the in vitro multipotential stem cell population (CFC-GEMM) from the cord blood sample greater than this lower limit might be arbitrarily considered appropriate for release for transplantation purposes.

### Example 3

### Measurement of Cell Potency

In order to measure cell potency, it is desirable to have a reference standard (RS) to which all samples can be compared. For the cell potency assays, the reference standard is a specific cell sample. In a cell potency assay kit, the reference standard may be provided with the kit as a vial of frozen cells. While the reference standard is arbitrary and would change from one kit lot to another, the assay is standardized against an external ATP standard, and the results, therefore, can be directly compared from one lot to another. Preferably, a 3-point cell dose response is performed with the reference standard with samples also assayed using at least the same 3-point cell dose response. If the cell dose response of the sample is not parallel with that of the reference standard, this indicates an error in preparing the sample cell dose response.

### Example 4

### Measuring Potency of a Drug

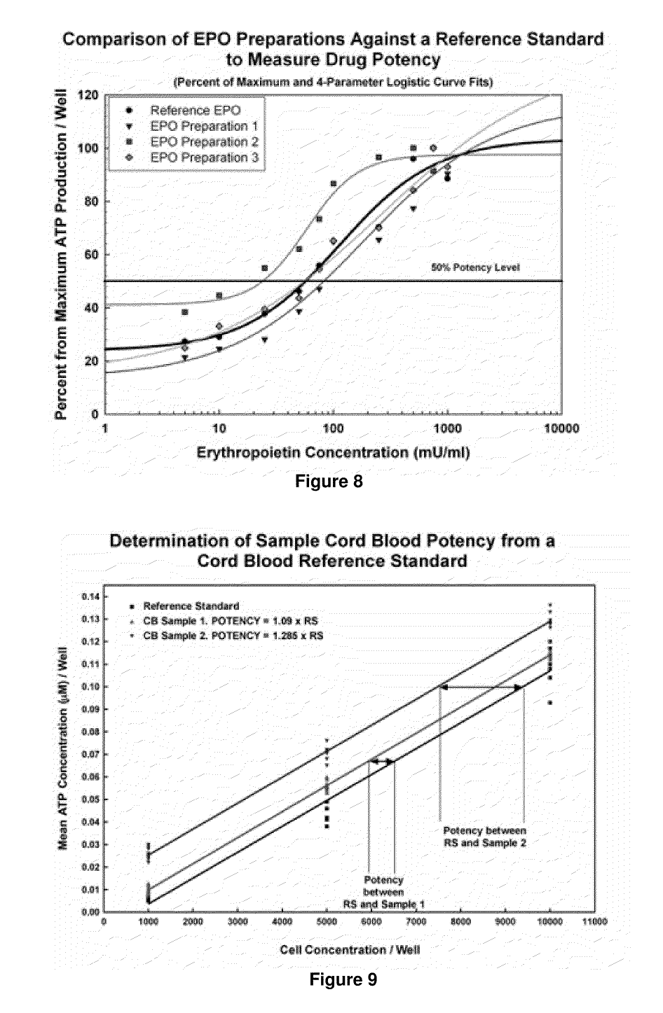

FIG. 8 shows the data for determining the potency of erythropoietin. An erythropoietin (EPO) reference standard and samples were serially diluted in medium over a 9-point dose response curve. Human bone marrow mononuclear cells were used as target cells at 5,000 cells/well. The cells and the appropriate doses of EPO were added to a HALO®-MeC assay, and 100 μl was dispensed into 8 replicate wells for each dose. The cultures were incubated for 7 days at 37° C. in a fully humidified atmosphere containing 5% CO₂ and 5% O₂. ATP concentrations were determined by luminescence on day 7. The ATP data was converted to percentages from the plateau dose response level (optimal EPO dose for each curve). The data was fitted to a 4-parameter logistic curve fit from which the EC50 values were obtained to estimate the potency of each of the EPO samples as compared to the reference standard.

In FIG. 8, the thicker solid black line (solid circles) represents the reference standard. In this case, the reference standard is a World Health Organization (WHO) standard to which all EPO samples are measured and compared. The three EPO preparations are shown by the thinner lines with the individual data points shown in triangles, squares, and diamonds. The linear portion of the curve is used to compare potency. Samples to the right of the reference standard have a lower potency and, therefore, less activity than the reference standard. Samples to the left of the reference standard have a greater potency and, therefore, more activity than the reference standard.

### Example 5

### Measuring Cell Potency of Cord Blood, Bone Marrow, or Mobilized Peripheral Blood for Transplantation Purposes

The same procedure may be used for measuring cell potency as was used with a drug in the previous example. In this example, random umbilical cord blood (UCB) mononuclear cells (MNC) from a single UCB unit frozen down at approximately 5,000 cell/well using an automated cryopreservation procedure were used as the UCB reference standard. Two UCB samples were obtained and cells were separated to produce a MNC fraction for each sample. For each sample, cells were counted and cell viability was determined. A vial of reference standard UCB was thawed and the cell count and viability were determined for the reference standard as well. The reference standard and sample UCB cell suspensions were diluted to final cell concentrations of 10,000, 5,000 and 1,000 cells/well. Cells were added to a HALO®-96 SEC platform kit using the proliferation agent mix specific for human CFC-GEMM cell. The mix contains EPO, GM-CSF, G-CSF, IL-3, IL-6, SCF, TPO and F13-L. (Available as CFC-GEMM 3 HALO® Master Mix from HemoGenix, Inc.). The cultures were incubated for 5 days at 37° C. in a fully humidified atmosphere containing 5% CO₂ and 5% O₂. The plates were then processed to determine the intracellular ATP concentration by bioluminescence measurements.

FIG. 9 shows the results of this experiment. Three-point cell dose response curves were analyzed using linear regression analysis and comparison of slope to ensure parallelism of straight lines. The horizontal difference between the reference standard and samples is determined at a 50% level. As illustrated in FIG. 9, both samples are displaced to the left of the reference standard and are, therefore, of higher cell potency and greater cellular activity than the reference standard.

### Example 6

### Cord Blood Potency and Release Testing

In this example, HALO®-96 SEC was used to study 56 cord blood samples for which the engraftment results were known. In this study, it was possible to demonstrate that the measured stem cell potency was directly related to the engraftment of the cord blood units. The development of a specific umbilical cord blood stem cell potency assay allows acceptance limits for release criteria for transplantation to be defined.

A. Materials and Methods

1. Umbilical Cord Blood Samples and Reference Standard

Fifty-six (56) umbilical cord blood (UCB) samples were provided by the University of Colorado Cord Blood Bank (a HERSA-Sponsored Cord Blood Bank). All samples tested were UCB vial samples that were cryopreserved at the same time as the original cord blood unit. The samples were chosen because the engraftment outcome for the samples were known. The conditions under which the samples were stored are shown in Table 2. Approximately 700 μl of each sample was available. The dates at which the samples were cryopresereved ranged over many years and, therefore, the cryopreservation procedure was not standardized between samples.

TABLE 2

| | | | | |
|---|---|---|---|---|
| Cells/Well | 2,500 | 5,000 | 7,500 | 10,000 |

Background Controls

| | | | | |
|---|---|---|---|---|
| No. of Cultures | 184 | 192 | 88 | 88 |
| Mean % CV | 5.0% | 4.3% | 3.7% | 3.4% |

CFC-GEMM

| | | | | |
|---|---|---|---|---|
| No. of Cultures | 184 | 192 | 88 | 88 |
| Mean % CV | 16.6% | 10.4% | 9.4% | 8.0% |

HPP-SP

| | | | | |
|---|---|---|---|---|
| No. of Cultures | 184 | 192 | 88 | 88 |
| Mean % CV | 12.0% | 9.7% | 6.7% | 5.7% |

Umbilical cord blood units for use as a reference standard (RS) were obtained from the University of Colorado Cord Blood Bank. These units had been rejected for storage on the basis of cell number and/or volume and would have been otherwise discarded. Permission to use all UCB samples and units for this study was provided by the Institutional Review Board (IRB) for the University of Colorado.

2. Umbilical Cord Blood Unit Reference Standard Cryopreservation

Each RS was processed individually for cryopreservation. The mononuclear cell (MNC) fraction of each unit was separated using NycoPrep 1.077 (Greiner Bio-One). The nucleated cell count and 3-part differential was determined using a Medonics blood analyzer, and the viability was determined using 7-aminoactinomycin D (7-AAD) by flow cytometry. The nucleated cell count was adjusted to 5,000 cells/ml and suspended in a mixture of Iscove's Modified Dulbecco's Medium (IMDM), 10% fetal bovine serum (FBS), and 7.5% dimethylsulphoxide (DMSO). The cells were cryopreserved in 1 ml aliquots using an automated liquid nitrogen rate-freezing procedure. The vials were stored in liquid nitrogen until used when they were processed using the same procedure as described below.

3. Umbilical Cord Blood Sample Pellet Thawing and Processing

The 56 test samples were stored in liquid nitrogen and chosen at random for assay. From two (2) to six (6) samples were processed at any one time. Each sample was rapidly thawed in a 37° C. water bath and transferred to a 50 ml sterile tube. Over a period of 10 minutes, a total of 10 ml UCB rehydration medium comprising IMDM with 10% bovine serum albumin (BSA) was added. Thereafter, 15 ml of NycoPrep 1.077 was underlayered, and the cells were centrifuged at room temperature for 20 minutes at 600×g. The interface cells were removed and transferred to another tube to which 20 ml IMDM was added. After another centrifugation for 10 minutes at 300×g, the supernatant was discarded and the cells were resuspended in IMDM. The nucleated cell count and viability was performed as described in the previous section.

Depending on whether sufficient cells were available to perform a 3-point cell dose response at 2,500, 5,000, and 10,000 cells/well, the order of priority for the cell populations detected was CFC-GEMM followed by HPP-SP. Similarly, if insufficient cells were available to perform a 3-point cell dose response, then the order of priority to perform a single assay at 5,000 cells/well was CFC-GEMM followed by HPP-SP.

4. Instrument-Based ATP Bioluminescence Proliferation Assay for the Determination of Umbilical Cord Blood Stem Cell Potency and Release Criteria

Intracellular ATP (iATP) is a biochemical marker, the concentration of which correlates directly with several important cell functionality markers. These markers include cell proliferation status/potential, cell number, and the cellular and mitochondrial integrity, which in turn provides the cell viability. When iATP is released from the cells by lysis, it acts as a limiting substrate for a highly sensitive luciferin/luciferase reaction that produces bioluminescence in the form of light. The light can be detected in a plate luminometer. The bioluminescence is in the form of a “glow” that is relatively stable and can be detected even after 30 minutes. The HALO® platform described herein incorporates a signal detection system for lympho-hematopoietic cells which has shown to be equivalent to and correlate with total colony counts from the colony-forming cell (CFC) assay (Reems et al., Transfusion, 48:620-628 (2008)). The assay used in this example did not incorporate methyl cellulose (MeC), but rather a liquid suspension expansion culture (SEC) to measure stem cell potency of cord blood samples. Since the SEC assay does not involve dispensing viscous MeC, the assay has several advantages over the MeC format. First, all reagents can be dispensed using normal pipettes that significantly reduces error. Second, because cell interaction can occur, the lag time to initiate proliferation is reduced, allowing the assay to be performed in only 5 days. Cell interaction also results in a two-fold increase in sensitivity. The assay has been designated HALO®-96 PQR (Potency, Quality Release) and was performed according to the manufacturer's instructions (HemoGenix, Inc., Colo. Springs, Colo.). The assay incorporates UCB reference standard (RS), which allows stem cell potency to be determined.

For those UCB samples that contained sufficient cells after thawing, a three-point cell dose response was performed with the final cell concentration/well at either 10,000 or 7,500, 5,000 and 2,500 cells/well. All cell doses were performed at eight replicates/dose. If sufficient cells were available, two stem cell populations were measured. The first was the in vitro multipotential, mature stem cell or CFC-GEMM (Colony-Forming Cell Granulocyte, Erythroid, Macrophage, Megakaryocyte) which, as its name implies, can produce cells of the hematopoietic lineages. The second was the more primitive High Proliferative Potential-Stem and Progenitor Cell (HPP-SP) that is capable of producing the CFC-GEMM, but also cells of both the hematopoietic and lymphopoietic lineages. These two stem cell populations have been shown to be highly predictive for the lympho-hematopoietic response and circulating mature elements (Rich, I. N. and Hall, K. M., Tox. Sci. 87:427-441 (2005)).

A master mix was prepared with the following components: 11.1 mg/ml bovine serum albumin (BSA), 0.055 mg/ml fetal bovine serum (FBS), 0.011 mg/ml recombinant insulin, and 0.222 mg/ml of iron saturated transferrin. In addition, alpha-thioglycerol is added so that the final concentration in culture is 0.1 mM. The master mixes and growth factors are made up in IMDM. Growth factors and cytokines specific for various cell populations are also added. Generally, for CFC-GEMM, the following growth factors and cytokines are used: EPO at 1-3 units/ml, GM-CSF at 10-20 ng/ml, G-CSF at 10-20 ng/ml, IL-3 at 10 ng/ml, IL-6 at 10-20 ng/ml, SCF at 20-50 ng/ml, TPO at 20-50 ng/ml, and Flt3-L at 20-50 ng/ml. For HPP-SP, IL-2 at 20-50 ng/ml and IL-7 at 20-50 ng/ml are also added in addition to the growth factors and cytokines used for CFC-GEMM.

Specifically, the CFC-GEMM master mix contained the following human recombinant growth factors and cytokines: erythropoietin (EPO, 3 U/ml), granulocyte-macrophage colony-stimulating factor (GM-CSF, 20 ng/ml), granulocyte colony-stimulating factor (G-CSF, 20 ng/ml), interleukin-3 (IL-3, 10 ng/ml), interleukin-6 (IL-6, 20 ng/ml), stem cell factor (SCF, 50 ng/ml), thrombopoietin (TPO, 50 ng/ml), and Flt-3 Ligand (Flt3-L, 10 ng/ml). For the stimulation of HPP-SP, IL-2 (20 ng/ml) and IL-7 (20 ng/ml) were also added. All growth factors and cytokines were purchased from CellGenix USA (Antioch, Ill.) or R & D Systems (Minneapolis, Minn.).

Once the culture master mix had been prepared, the contents of each tube were mixed by vortexing and 100 μl was dispensed into each replicate well using an electronic, positive displacement repeater pipette. Cultures were incubated in a 37° C. incubator at an atmosphere of 5% CO₂ and 5% O₂. Periodically, during the course of this study, a vial of RS cord blood cells was thawed (as described above) and assayed in parallel with one or more UCB samples being tested.

After five days in culture, 100 μl of an ATP monitoring reagent (ATP-MR) was added to each well using a multi-channel pipette or liquid handler, and the contents of each well were mixed. After a ten minute incubation at room temperature, the bioluminescence of each well was measured using a LMax Plate Luminometer (Molecular Devices, Inc., Ramsey Minn.). The readout of the luminometer was expressed as relative luminescence units (RLU).

5. The ATP Standard Curve

Performing an ATP standard curve allows standardization of the assay. The ATP standard curve is used to calibrate the instrument and ensure that the reagents are working correctly, and also provides an indication of “pipetting efficiency”. In addition, since luminometers from different manufacturers produce different ranges of relative luminescence unit (RLU) readouts, the ATP standard curve allows non-standardized RLU values to be automatically converted to standardized ATP concentrations (μM). An ATP standard curve was performed prior to measuring bioluminescence for the UCB samples. Because the assay is standardized each time it is performed, multiple experiments performed over various time periods can be directly compared with each other.

The ATP standard curve was performed by dispensing 100 ml of 1 mM, 0.5 mM, 0.1 mM, 0.05 mM and 0.01 mM ATP standards in 4 replicate wells of a 96-well plate. In addition, four 100 μl replicates containing just IMDM were also dispensed and used to determine the ATP background. In addition, four replicates each of a high and low control were also included. To each well, 100 μl of ATP-MR was added. The contents were mixed, and after a two minute incubation at room temperature, the plate was read in a plate luminometer.

6. Calculations and Statistics

The luminometer software (SoftMax Pro, Molecular Devices, Sunnyvale, Calif.) was programmed to produce individual RLU and ATP values, mean, standard deviations and percent coefficients of variation (% CV) of each of the groups. These results were then exported to Microsoft Excel for further calculations and statistics analysis.

Three-point cell dose response curves for both the reference standard and the UCB samples were subjected to linear regression analysis. The goodness of fit or coefficient of determination (r²) and the slope of the line were calculated. The slopes of the UCB samples were then compared to that of the reference standard using Prism software, version 5 (GraphPad Software, La Jolla, Calif.). If the sample dose response curves were statistically parallel to the RS, a common slope could be calculated and the stem cell potency ratio calculated from the horizontal displacement compared to the RS. The potency of the RS was designated to be 1. In the majority of cases, however, parallelism of the samples to the RS was not obtained. In this case, the slope of the sample cell dose response was then compared to that of the RS in order to calculate the potency ratio.

Correlation was performed using either Pearson (parametric) or Spearman (non-parametric) statistics and the correlation coefficients (R), together with significance values (P) were calculated either in a pairwise or in a ranked fashion, respectively. In some cases, linear regression analysis was also accompanied by the 95% confidence and predictive limits. Where appropriate, Student's t-test was used to determine whether differences in proliferation responses between cell populations were statistically significant. All statistical calculations were performed using SigmaStat software, version 3.1 (Systat Software, Richmond, Calif.).

B. Results

1. Assay Precision

Part of the validation procedure was to determine assay precision or reproducibility. The assays involved in this example involved determining the proliferation of cells cultured in the absence of growth factors (background control), and those cultured to assess CFC-GEMM and HPP-SP proliferation. As shown in Table 2, the background and each of the stem cell populations were tested at four different cell concentrations of eight replicates each over a period of three months. For the lower cell concentrations (2,500 and 5,000 cells/well), a total of 184 or 192 wells were assessed. For the higher concentrations (7,500 and 10,000 cells/well), 88 wells were assessed. The results indicate that at low concentrations, the percent coefficients of variation (% CV) was, as expected, higher, but the greatest CV was less than 17%.

2. Umbilical Cord Blood Sample Information

All UCB samples tested were contained in cryopreservation vials. Thirty-two of the fifty-six samples were stored at −80° C. The remaining twenty-four samples were stored in liquid nitrogen (LN₂). The samples were obtained from the larger UCB units, and all fifty-six UCB units were stored in LN₂. Although seventeen out the thirty-two UCB units actually engrafted, all of the thirty-two UCB samples that were stored at −80° C. and assayed produced ATP levels lower than the arbitrary limit (see below) of 0.04 μM. In the majority of cases, no ATP production and, therefore, no proliferation, was detected. This implies that a change in storage freezing temperature could hamper or even eliminate the proliferation potential of the UCB stem cell cells.

The total nucleated cell count (TNC), viability, CD34, and engraftment information was also determined for all 56 UCB samples. The shortest period from cryopreservation to thawing for transplantation was 91 days, while the longest period a sample had been frozen prior to use was 9 years and 311 days. Forty-one, or 73% of the samples, engrafted with a minimum time to greater than 500 absolute neutrophil count (ANC)/μl of 5 days and a maximum time of 114 days. The minimum time to a platelet count of 50,000/μl or greater was 2 days, and the maximum was 237 days.

3. Relationship between TNC, viability, CD34 and ATP Concentration

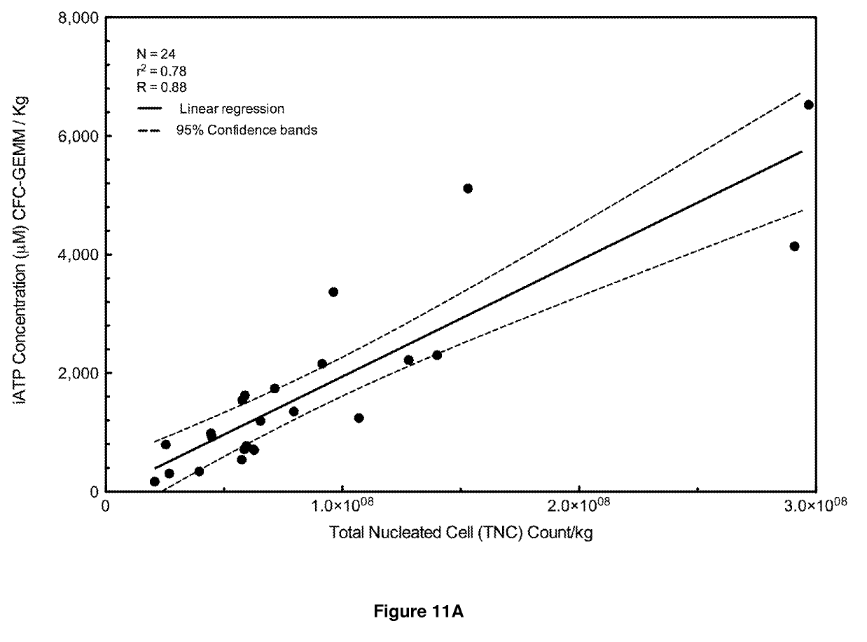

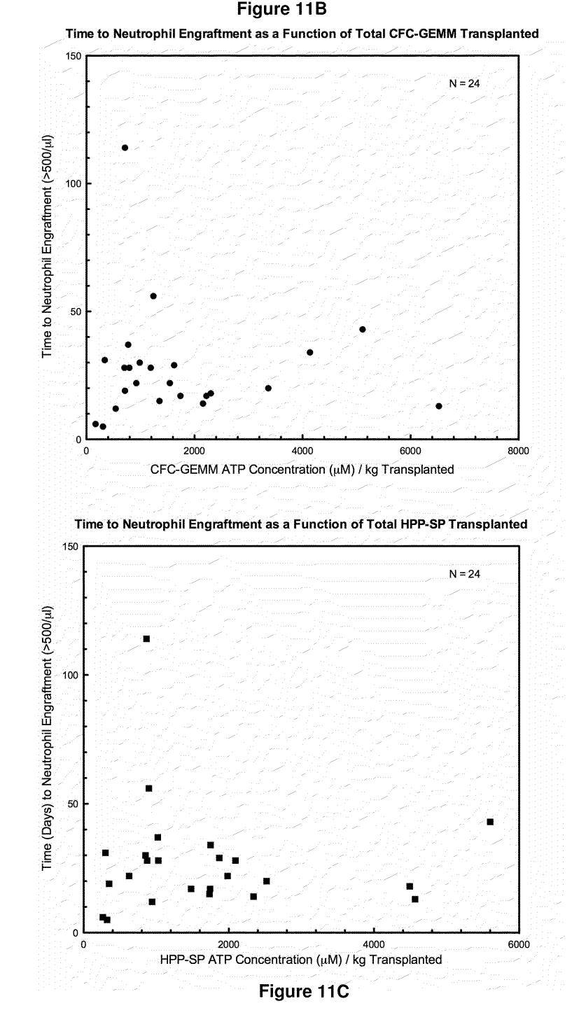

For the 24 UCB samples that were stored in LN₂ and could be analyzed with respect to potential statistical relationships between TNC, viability, CD34, and ATP concentrations, the only pair of observations that produced a significant correlation was between the TNC/kg and ATP/kg as shown in FIG. 11. All other combinations of parameters, including time to neutrophil (>500 cells/μl) and platelet (>50,000/μl) engraftment, produced no significant correlations.

4. Determination of Stem Cell Potency

In addition to an appropriate and validated assay to measure cell potency, a reference standard and a dose response curve are utilized in order to determine cell potency. The standard procedure for estimating potency is to compare a dose response for a sample with that of a RS of the same material. One expected result would be that the linear section of the dose response curve would be parallel to that of the RS. If the sample response is displaced to the left or right of the RS, the sample has a higher or lower potency, respectively. If the dose response lines are statistically parallel to each other, a horizontal line drawn anywhere from the Y-axis that bisects the dose response curve would provide the same estimation of potency when read off the X-axis. The potency ratio of the sample can then be calculated by dividing the sample X-axis value by the reference standard X-axis value.

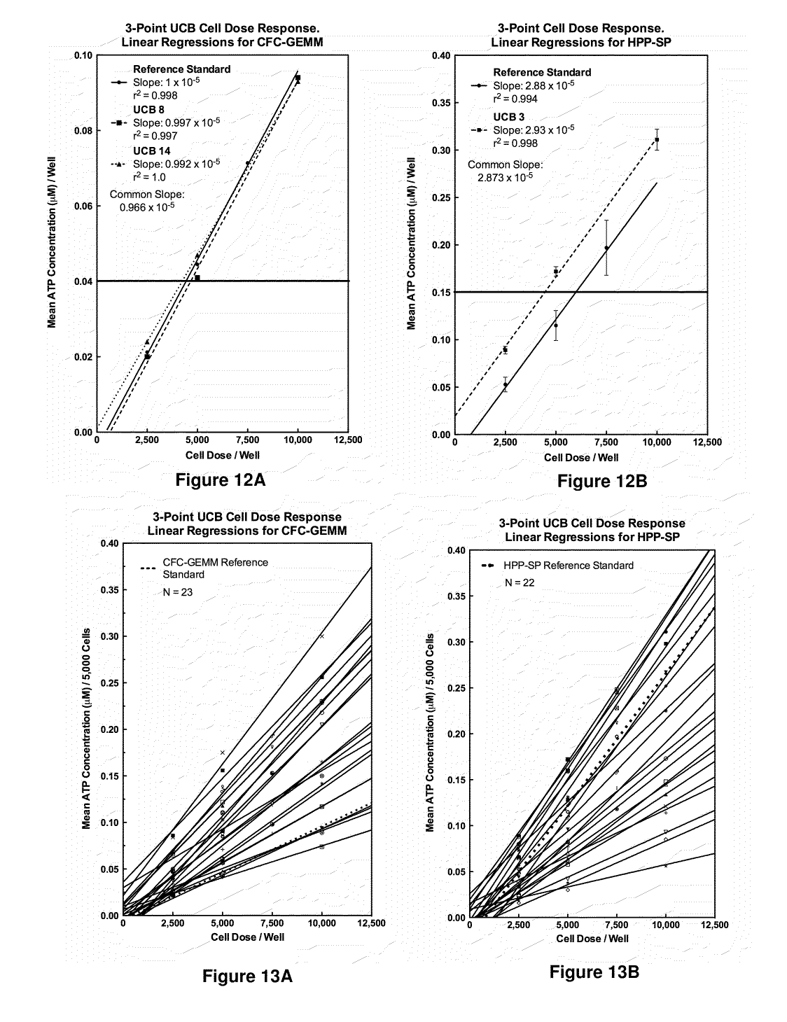

FIGS. 12A and 12B show that, out of the twenty-four UCB samples stored in LN₂, only three produced statistically parallel dose response lines to the RS for either CFC-GEMM (samples 8 and 14, FIG. 12A) or HPP-SP (sample 3, FIG. 12B). For both cell populations, a common slope could be calculated. For samples 8 and 14, there was hardly any displacement to the left or right of the RS, indicating that the CFC-GEMM potency of these 2 samples was essentially the same as that for the RS, i.e. 1. In contrast, sample 3 was displaced to the left of the RS for HPP-SP, so the potency of this sample was greater than that of the RS with a potency ratio of approximately 1.5. When the linear regressions of the cell dose response curves are parallel to the RS, the samples contain a greater or lower number of stem cells, but the “stemness” or “primitiveness” of the stem cells is similar to that in the RS.

FIG. 12A shows the 3-point cell dose response for CFC-GEMM from the reference standard (RS) and UCB samples 8 and 14 that are statistically parallel (95% confidence limits) to each other over the dose range measured. The slope for RS was 1×10⁻⁵ (r²=0.998, P<0.001). The slope for UCB sample 8 was 0.997×10⁻⁵ (r²=0.997, P <0.001). The slope for UCB 14 was 0.992×10⁻⁵ (r²=1.0). The common slope for the RS and both UCB samples was 0.966×10⁻⁵. FIG. 12B shows the 3-point cell dose response for HPP-SP from the reference standard (RS) and UCB sample 3 that are statistically parallel (95% confidence limits) to one another over the measured dose range. The slope for RS was 2.88×10⁻⁵ (r²=0.994, P<0.001). The slope for UCB 3 was 2.93×10⁻⁵ (r²=0.998, P<0.001). The common slope was 2.92×10⁻⁵. The individual points from which the linear regressions were derived are the mean of eight replicates ± standard deviations.

The remaining UCB samples, stored in LN₂, were not parallel to the respective RS, but demonstrated different slopes compared to the RS as shown in FIGS. 13A and 13B. For FIG. 13, all twenty-five UCB samples were included with the RS (dotted line) for CFC-GEMM. Twenty-three of the samples were tested for the presence of HPP-SP as well as CFC-GEMM. These results illustrate that stem cells exhibit different degrees of “stemness” and, therefore, different potentials for proliferation. The greater the overall proliferation potential of the sample, the steeper the slope of the dose response curve. If a vertical line is placed at any cell concentration on the X-axis, the bisecting line would indicate that, as the iATP concentration increases, the slope of the cell dose response also increases.

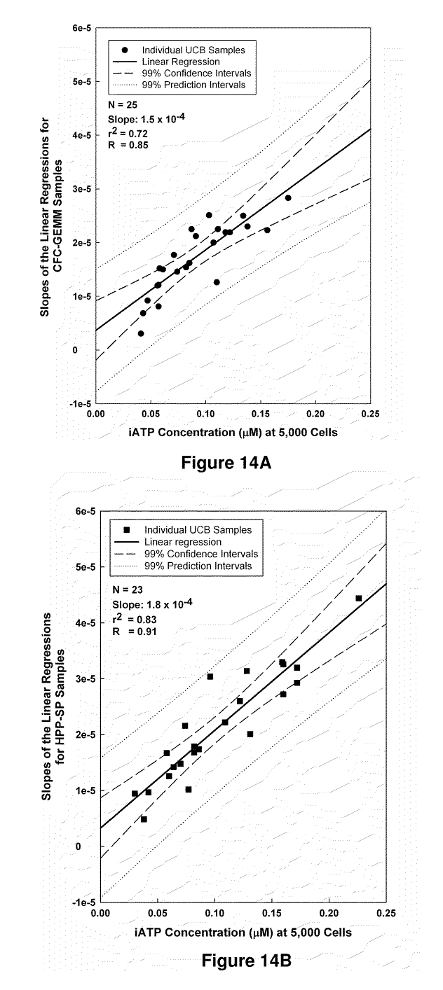

FIGS. 14A and 14B show the correlation between the iATP concentration and the slope of the linear regression curves for cord blood samples that did not exhibit parallelism with the reference standard. There is a direct correlation between the iATP concentration and slope of the dose response line for both the CFC-GEMM population, shown in FIG. 14A, and that of the HPP-SP population, shown in FIG. 14B. Furthermore, the slope of the resulting linear regressions for each of these correlations shows that the slope for HPP-SP (1.8×10⁻⁴) is greater than that for CFC-GEMM (1.5×10⁻⁴), which is indicative of the fact that HPP-SP is a more primitive stem cell population than the CFC-GEMM. FIGS. 14A and 14B illustrate that there is a correlation between the iATP concentration for both CFC-GEMM and HPP-SP, and shows the slope of the three-point cell dose response linear regression curve for each of these populations. The slope is greater for HPP-SP than for CFC-GEMM, indicating a greater proliferation potential. The solid linear regression line for both graphs is bounded by the 99% confidence and 99% predictive intervals.

5. Relationship Between iATP Concentration, Release Criteria and Stem Cell Potency

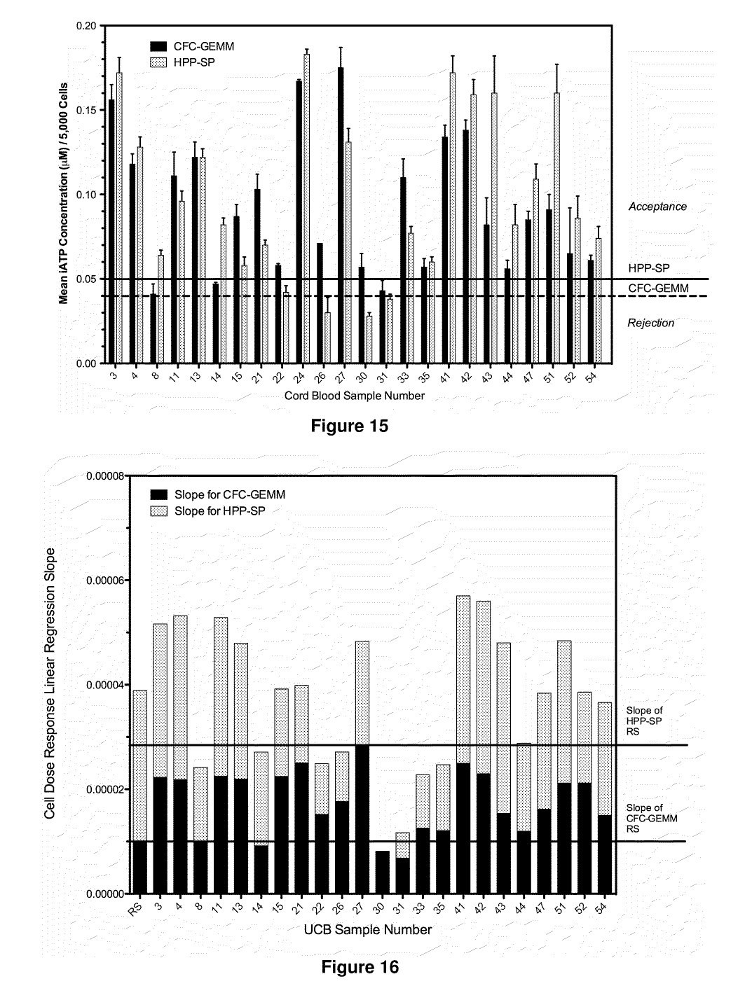

FIG. 15 shows the iATP concentrations for both CFC-GEMM and HPP-SP at 5,000 cells/well (5×10⁵ cells/ml) for all samples stored in LN₂. From these and other results (not shown), arbitrary acceptance/rejection values have been assigned for CFC-GEMM at 0.04 μM and for HPP-SP at 0.05 μM. Below these values, a sample would be rejected. For all UCB samples stored at −80° C., all iATP concentrations for both CFC-GEMM and HPP-SP were below this arbitrary level. As seen in FIG. 15, some of the CFC-GEMM samples might also have been rejected (samples 8 and 31) as well as some HPP-SP samples (samples 22, 26, 30, and 31). However, a stem cell product does not contain just one cell population, but a pool of stem cell populations.

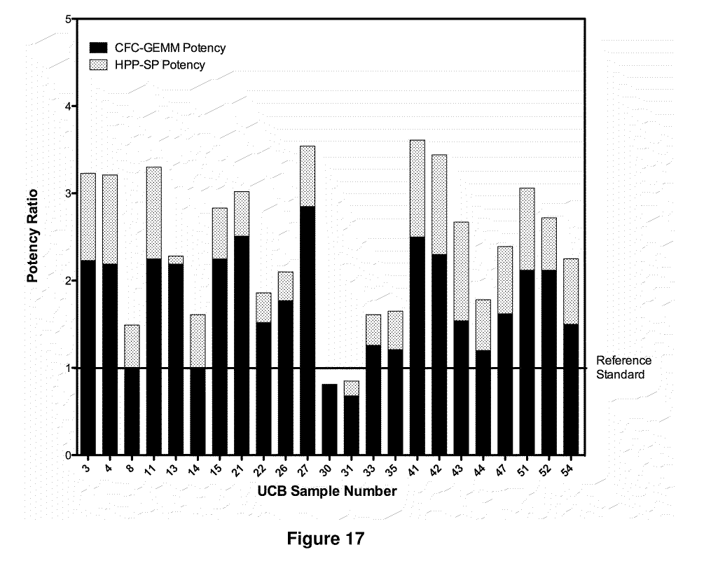

Despite the fact that a single UCB reference standard is assessed as a RS for both CFC-GEMM and HPP-SP, as seen in FIG. 14 and FIG. 16, it is the combination of both populations and, therefore, the combined slope that determined the overall proliferation potential of the UCB unit. FIG. 16 shows the cell dose response linear regression slope for each of the CFC-GEMM and HPP-SP samples. Thus, the cumulative iATP concentration and slope produces the cumulative stem cell potency, shown in FIG. 17. The cell potency of a specific stem cell population is calculated by dividing the slope of the cell sample dose response curve by the slope of the reference standard dose response curve. This gives the “potency ratio”. The slope of the reference standard is designated as 1. Therefore, if the potency ratio is greater than 1, the stem cell population has a greater potency than that of the reference standard, and visa versa. The steeper the slope of the stem cell dose response curve, the more primitive the stem cell population, the greater the proliferation potential, and the greater the stem cell potency. The greater the potency, the higher the probability that the stem cell product will engraft, although engraftment itself is dependent upon the patient. As seen in FIG. 17, CFC-GEMM contributes the major portion of the potency determination. This is to be expected because the HPP-SP population contains fewer stem cells that are more primitive and mostly quiescent (out of cell cycle) in nature, but when induced into cell cycle, demonstrate a greater proliferation potential than CFC-GEMM. The RS always has a potency of 1. Sample 30 did not contain sufficient cells to perform a HPP-SP assay. However, both stem cell populations could be assayed in UCB sample 31, and this sample still produced a potency less than the RS. Despite this result, all twenty-four UCB samples engrafted.

The study described in this example presents a standardized and reproducible, non-subjective, in vitro assay that has the potential (1) to distinguish between cord blood units that will growth and those that may not, (2) to help define acceptance limits for release criteria, (3) to measure stem cell potency as defined by regulatory agencies, and (4) to help predict engraftment. The results also demonstrate that there is an intimate relationship between the iATP concentration, which defines the slope of a 3-point cell dose response curve and the potency of the stem cell product.

This study demonstrates that stem cell potency, defining acceptance and rejection criteria for cord blood units destined for transplantation into patients, and the ability to predict engraftment potential can be performed with a single, rapid, easy to use, instrument-based ATP bioluminescence assay that exhibits the properties and characteristics required by regulatory agencies. The cell potency assays provided are particularly useful for determining the proliferative capacity and, therefore, the potency, of stem cells and cord blood used for transplantation. The cell potency assays for stem cell transplantation and cord blood storage take advantage of the fact that cell proliferation and differentiation are two separate and distinct, although related, processes. By taking advantage of these different processes, it has been possible to design a cell potency assay that is non-subjective, that is standardized, and that can be subjected to validation and proficiency testing.

Any patents or publications mentioned in this specification are indicative of the level of those skilled in the art to which the invention pertains. Further, these patents and publications are incorporated by reference in their entireties.

The present examples are to be considered as illustrative and not restrictive, and the invention is not to be limited to the details given herein, but may be modified within the scope of the appended claims.

1. An assay method for determining the potency of a population of primitive lympho-hematopoietic cells, the method comprising the steps of:
(a) incubating a cell population comprising primitive lympho-hematopoietic cells in a cell growth medium comprising fetal bovine serum having a concentration of between 0% and about 30%, and in an atmosphere having between about 3.5% oxygen and about 7.5% oxygen;
(b) contacting the primitive lympho-hematopoietic cell population with a proliferation agent, the proliferation agent comprising one or more growth factors, one or more cytokines, or combinations thereof;
(c) contacting the primitive lympho-hematopoietic cell population with a reagent capable of reacting with ATP and generating luminescence in the presence of ATP; and
(d) detecting luminescence generated by the reagent that reacted with the ATP in the primitive lympho-hematopoietic cell population, the level of luminescence indicating the amount of ATP in the primitive lympho-hematopoietic cell population, wherein the amount of ATP indicates the proliferative capacity and, therefore, the potency of the primitive lympho-hematopoietic cells.

2. The method of claim 1, wherein the primitive lympho-hematopoietic cells are derived from bone marrow cells, umbilical cord blood cells, or mobilized peripheral blood.

3. The method of claim 1, wherein the concentration of fetal bovine serum is between about 0% and 10%.

4. The method of claim 1, wherein the concentration of oxygen in the atmosphere is about 5%.

5. The method of claim 1, wherein the reagent capable of reacting with ATP and generating luminescence in the presence of ATP comprises luciferin and luciferase.

6. The method of claim 1, wherein the cell growth medium further comprises methyl cellulose having a concentration of between about 0.4% and about 0.7%.

7. A high-throughput assay method for rapidly identifying a population of primitive lympho-hematopoietic cells having a potency suitable for transplantation into a patient, comprising the steps:
(a) providing a cell population comprising primitive lympho-hematopoietic cells;
(b) incubating the cell population in a cell growth medium comprising a concentration of fetal bovine serum between 0% and 30% and in an atmosphere having between about 3.5% oxygen and 7.5% oxygen;
(c) contacting the cell population with at least one proliferation agent selected from the group consisting of erythropoietin, granulocyte-macrophage colony stimulating factor, granulocyte colony stimulating factor, macrophage colony stimulating factor, thrombopoietin, stem cell factor, interleukin-1, interleukin-2, interleukin-3, interleukin-6, interleukin-7, interleukin-15, Flt3L, leukemia inhibitory factor, insulin-like growth factor, insulin, and combinations thereof;
(d) contacting the cell population with a reagent capable of generating luminescence in the presence of ATP; and
(e) detecting luminescence generated by the reagent contacting the cell population, the level of luminescence indicating the proliferative status of the primitive lympho-hematopoietic cells, and wherein the proliferative status of the primitive hematopoietic cells indicates the potency of the cell population and suitability of the cell population for transplantation into a recipient patient.

8. The method of claim 7, wherein the primitive lympho-hematopoietic cells are derived from umbilical cord blood cells, bone marrow cells, or mobilized peripheral blood.

9. The method of claim 7, wherein the concentration of fetal bovine serum is between about 0% and 10%.

10. The method of claim 7, wherein the concentration of oxygen in the atmosphere is about 5%.

11. The method of claim 7, wherein the reagent capable of reacting with ATP and generating luminescence in the presence of ATP comprises luciferin and luciferase.

12. The method of claim 7, wherein step (d) further comprises defining a threshold level of ATP, wherein the cell population is released for transplantation into a recipient patient only if the ATP level of the cell population is above the threshold.

13. An assay method for determining the potency of a cell population for use in a cell-based therapy, the method comprising the steps of:
(a) incubating a sample cell population and a reference standard from the cell population in a dose-dependent manner in a cell growth medium comprising fetal bovine serum having a concentration of between 0% and about 30%, and in an atmosphere having between about 3.5% oxygen and about 7.5% oxygen;
(b) contacting the sample cell population with a proliferation agent, the proliferation agent comprising one or more growth factors, one or more cytokines, or combinations thereof;
(c) contacting the sample cell population and the reference standard with a reagent capable of reacting with ATP and generating luminescence in the presence of ATP; and
(d) detecting luminescence generated by the reagent that reacted with the ATP in the sample cell population and the reference standard, the level of luminescence indicating the amount of ATP in the sample cell population and the reference standard;
(e) generating a dose response curve for the sample cell population and the reference standard, and comparing the level of ATP in the sample cell population and the reference standard to determine the potency of the sample cell population relative to the reference standard.

14. The method of claim 13, wherein the cell population further comprises umbilical cord blood cells, bone marrow cells, or mobilized peripheral blood.

15. The method of claim 13, wherein the cell population further comprises Colony-Forming Cell Granulocyte, Erythroid, Macrophage, Megakaryocyte (CFC-GEMM) cells or High Proliferative Potential-Stem and Progenitor Cell (HPP-SP) cells.

16. The method of claim 13, wherein the concentration of fetal bovine serum is between about 0% and 10%.

17. The method of claim 13, wherein the concentration of oxygen in the atmosphere is about 5%.

18. The method of claim 13, wherein the reagent capable of reacting with ATP and generating luminescence in the presence of ATP comprises luciferin and luciferase.

19. The method of claim 15, wherein the cell population comprises Colony-Forming Cell Granulocyte, Erythroid, Macrophage, Megakaryocyte (CFC-GEMM) cells, and the proliferation agent comprises erythropoietin (EPO), granulocyte-macrophage colony-stimulating factor (GM-CSF), granulocyte colony-stimulating factor (G-CSF), interleukin-3 (IL-3), interleukin-6 (IL-6), stem cell factor (SCF), thrombopoietin (TPO), and Flt-3 Ligand (Flt3-L).

20. The method of claim 15, wherein the cell population comprises of High Proliferative Potential-Stem and Progenitor Cell (HPP-SP) cells, and the proliferation agent comprises erythropoietin (EPO), granulocyte-macrophage colony-stimulating factor (GM-CSF), granulocyte colony-stimulating factor (G-CSF), interleukin-3 (IL-3), interleukin-6 (IL-6), stem cell factor (SCF), thrombopoietin (TPO), Flt-3 Ligand (Flt3-L), interleukin-2 (IL-2), and interleukin-7 (IL-7).
